(19) United States
(12) Patent Application Publication
(10) Pub. No.: US 2019/0006032 A1
(43) Pub. Date: Jan. 3, 2019

(54) INTERVENTIONAL MEDICAL REPORTING APPARATUS

(71) Applicant: KONINKLIJKE PHILIPS N.V., EINDHOVEN (NL)

(72) Inventors: ALEXANDRA GROTH, HAMBURG (DE); IVO MATHIEU JACOBUS CANJELS, BUDEL (NL); VINCENT MAURICE ANDRE AUVRAY, MEUDON (FR); JUERGEN WEESE, NORDERSTEDT (DE); RAOUL FLORENT, VILLE D'AVRAY (FR)

(21) Appl. No.: 15/780,431

(22) PCT Filed: Dec. 20, 2016
(86) PCT No.: PCT/EP2016/081849

§ 371 Date: May 31, 2018

(30) Foreign Application Priority Data
Dec. 30, 2015 (EP) 15307180.8

(51) Int. Cl. G16H 15/00; G16H 10/60; G16H 30/20

(52) U.S. Cl. CPC G16H 15/00; G16H 30/20; G16H 10/60

(57) ABSTRACT

Interventional medical procedures involve a complex sequence of actions, often involving many different medical professionals and items of equipment. These actions, and their notable attributes, should be recorded in detail, in case they are required during a consultation in the future. A typical medical interventional procedure could require hundreds, or even thousands, of actions to be recorded. This places demands on a medical practitioner during the intervention. Typically, important information is dictated into a report after an intervention. Attempting to produce the report by traditional means, such as by dictation, after the intervention is time-consuming. The proposed approach enables the formation of clinical reports based on structured interventional medical reporting data recorded during a medical intervention in an easier, and more accurate way. Interventional procedure status indications are used which may be derived from existing medical equipment, or information inputs.

### FIELD OF THE INVENTION

The present invention relates to an apparatus for the formation of a clinical report during an interventional medical procedure using structured interventional medical reporting data, a method for the formation of a clinical report of an interventional medical procedure using structured interventional medical reporting data, a system, a computer program element, and a computer readable medium.

### BACKGROUND OF THE INVENTION

After a medical intervention, a physician must complete a patient report. The patient report contains a range of information, such as the findings and the outcome of the intervention procedure. For example, after a typical catheterization procedure, information in the report could comprise patient identifiers, the procedure date, the name of the physician, and then a list of procedure-specific parameters. In the case of a catheterization, such quantities comprise medication provided to the patient during the procedure, graphics relevant to the catheterization, doses of anaesthesia or other chemicals, vital signs and haemodynamic support.

It will be appreciated, that inserting these quantities into a report manually, or via dictation, is time consuming. Recording the final report using the traditional technique of dictation also suffers from the lack of lexical standardization between physicians.

Therefore, intra-procedural medical reporting approaches can be further improved. The document “Health Policy Statement on Structured Reporting for the Cardiac Catheterization Laboratory”, a report of the American Quality Committee, by Sanborn et al., published in the Journal of the American College of Cardiology, vol. 63, no. 23, 2014, ISSN 0735-1097 discusses issues surrounding medical reporting.

### SUMMARY OF THE INVENTION

It would be advantageous to have an improved technique for providing intra-procedural reporting during interventional medical procedures.

The object of the present invention is solved by the subject-matter of the independent claims, wherein further embodiments are incorporated in the dependent claims.

A first aspect of the invention provides an apparatus for the formation of a clinical report during an interventional medical procedure using structured interventional medical reporting data. The apparatus comprises:

a processing unit

The processing unit is configured to receive a report template class of an interventional medical procedure. The report template class comprises a plurality of template class elements, and a plurality of template class rules. The apparatus is further configured to receive an interventional procedure status indication from an interventional procedure status monitoring device, wherein the interventional procedure status indication provides information about a current stage of a plurality of stages of the interventional medical procedure.

The apparatus is configured to determine an availability condition of a template class element in the plurality of template class elements by comparing the interventional procedure status indication to one of the plurality of template class rules, to enable a data input into the template class element if the availability condition is satisfied, to receive data into the template class element for which data input has been enabled to form structured interventional medical reporting data, to generate a clinical report of the interventional medical procedure using structured interventional medical reporting data, and to output the clinical report.

Accordingly, an apparatus is provided enabling interventional medical data to be provided in the form of a clinical report from structured interventional medical reporting data acquired throughout the duration of an interventional medical procedure, in a manner which is flexible to changes which can occur during the interventional medical procedure, and in a manner which is convenient to a medical professional.

Another view is that an apparatus according to the first aspect enables an interventional procedure status indication to be derived from a wide range of technical inputs related to the intervention, or a patient undergoing such an intervention. For example, data can be provided from the monitoring of the patient by medical monitoring equipment such as an ECG machine. The interventional procedure status indication may enable the current phase of a medical procedure to be tracked by the apparatus, and for information into certain report template classes to be enabled accordingly.

Optionally, the data input into the template class element is enabled during the interventional procedure. In other words, the stage of the intervention is tracked, and different data may be entered dependent on the point within the procedure that has been reached. According to an embodiment of the first aspect, an apparatus is provided according to the first aspect, wherein the processing unit is further configured to display, on a graphical user interface, a graphical input element linked to the template class element. The graphical input element is selected from a plurality of graphical input elements. The data is received into the template class element via the graphical input element.

Accordingly, it is possible to form structured interventional medical reporting data using a graphical user interface displayed on a screen of an item of medical equipment.

For example, during a catheterization, a physician will use equipment commonly found in a catheterization laboratory, including a C-arm imaging setup, and vital signs monitoring equipment. An interface of the catheterization lab equipment can, thus, be adapted for convenient data input.

According to an embodiment of the first aspect, there is provided an apparatus as previously described, wherein the processing unit is further configured to receive patient parameter information from a patient monitoring device. The apparatus is further configured to pre-populate the graphical input element with an initial data value taken from the patient parameter information. The initial data value of the template class element in the graphical input element is previewed, and a user confirmation command confirming the initial data value as a final data value is received. The apparatus is configured to store the final data value into the template class element as structured interventional medical reporting data.

Accordingly, an apparatus is provided in which data values automatically populate input fields of a graphical input element on a graphical user interface. This removes from a physician the burden of having to read relevant values from medical equipment, such as O₂ saturation, because, instead, the values are automatically populated into the fields.

Therefore, important report parameters can be provided in the structured interventional medical reporting data in an error-free manner.

According to an embodiment of the first aspect, an apparatus is provided as previously described according to the first aspect, wherein the processing unit is further configured to receive standardized vocabulary data linked to the template class element. The standardized vocabulary data comprises a plurality of vocabulary elements. Each vocabulary element represents a standard medical term.

The apparatus according to this embodiment is further configured to select a subset of vocabulary elements from the plurality of vocabulary elements based on the template class element, to display a subset of vocabulary elements on the graphical user interface as the graphical input element, and to enable a selection of a selected vocabulary element, out of the subset of vocabulary elements, and to receive the selected vocabulary element into the template class element.

Accordingly, a standardized vocabulary may be displayed to a medical professional, and provided in the form of a selection, to ensure that a medical professional imports words chosen from a standardized lexicon when composing a medical report. This enables a medical professional to complete a medical report using a standardized lexicon without, for example, having to wait to look up standard words during a medical procedure.

According to an embodiment of the first aspect, an apparatus is provided as described, wherein the processing unit is further configured to store a sequence of interventional procedure status indications, to detect the end of a current stage of the interventional medical procedure by comparing the sequence of interventional procedure status indications to an interventional procedure stage rule, and to display the graphical input element on the graphical user interface if the end of the current stage of the interventional medical procedure has been detected.

Accordingly, an input of medical reporting data may be delayed until an intermediate point in an interventional procedure, which is deemed to be convenient for inputting medical data. For example, during the positioning of a stent using a balloon, it may be desirable to record the final location of the stent. It is not convenient to do this during the approach of a catheter to the implantation location, or during balloon expansion, or during catheter removal, because these operations require the full attention of the physician. Thus, according to this embodiment, the apparatus tracks the phases of the stent implantation operation, and prompts a user for data input at a convenient time, such as when the stent has been fitted, and the balloon has been withdrawn.

According to an embodiment of the first aspect, an apparatus is provided as described previously, wherein the processing unit is further configured to acquire a plurality of medical images from an interventional medical imaging device, to display the plurality of medical images using the graphical user interface, to enable a user selection of a medical image of the plurality of medical images, and to store user-selected medical images in a template class element of the structured interventional medical reporting data.

Accordingly, an existing interventional medical imaging device may be used to load medical images into template classes of the structured interventional medical reporting data, for possible subsequent use in a medical report. By displaying the plurality of medical images using the graphical user interface, the user can choose an image to be inserted into the report more easily.

According to an embodiment of the first aspect, an apparatus is provided as described previously, wherein the processing unit is further configured to receive a second plurality of interventional medical images from an interventional medical imaging device, and to automatically select a representative image from the second plurality of medical images based upon an interventional medical image selection algorithm to provide a selected representative interventional image,

wherein a data input into the template class element comprises enabling the input of the selected interventional representative image into the template class element.

Accordingly, a suitable medical image can be selected from a sequence, and inserted into a template class element of the structured interventional medical reporting data, for potential future use in a medical report, with no or little user intervention. This has the effect of reducing the distraction of a medical professional during an interventional procedure.

According to an embodiment of the first aspect, the interventional procedure status indication is a signal received by the processing unit resulting from the group of signals:

(i) a stent deployment control signal;

(ii) a stent deployment visual detection signal;

(iii) a device positioning recognition signal;

(iv) a haemodynamic measurement stage signal;

(v) an electrophysiological measurement stage signal;

(vi) a pressure measurement signal;

(vii) a contrast injection signal;

(viii) a medical image state signal;

(ix) an electrocardiogram signal;

(x) a balloon inflation signal;

(xi) a C-arm orientation parameter;

(xii) a manual procedure status input; and

(xiii) a catheter positioning recognition signal.

According to an embodiment, the interventional procedure status indication may comprise any combination of the elements in the group of the previous paragraph.

Accordingly, a wide range of stimuli may be used by the processor to derive a interventional procedure status which can be used to activate different template class elements for information input. For example, the presence of a certain C-arm orientation parameter in terms of azimuth and inclination angle, can be used to infer that either a left or a right-hand side cardiac image is being taken. Accordingly, data input into a template class element referring to a left or a right cardiac examination can be activated, to enable the appropriate type of cardiac information to be filled in to the appropriate type of cardiac information form.

According to an embodiment of the first aspect, an apparatus is provided as described previously, wherein the processing unit is further configured to generate an interventional medical procedure report using the structured interventional medical reporting data by transferring the data stored in the plurality of template class elements into the report template class, and to output the interventional medical procedure report.

Accordingly, a comprehensive and accurate report may be automatically assembled using the structured interventional reporting data. This results in the automatic production of accurate medical reports characterising an intervention in a very short time.

According to a second aspect of the invention, a method for the formation of a clinical report of an interventional medical procedure using structured interventional medical reporting data, comprising the following steps:
a) receiving a report template class of an interventional medical procedure,

wherein the report template class comprises a plurality of template class elements, and a plurality of template class rules,
b) receiving an interventional procedure status indication from a procedure status monitoring device,

wherein the interventional procedure status indication provides information about a current stage of a plurality of stages of the interventional medical procedure;
c) determining an availability condition of a template class element in the plurality of template class elements by comparing the interventional procedure status indication to at least one of the plurality of template class rules;
d) enabling a data input into the template class element if the availability condition is satisfied;
e) receiving data into the template class element for which data input has been enabled to form structured interventional medical reporting data; and
f) generating a clinical report of the interventional medical procedure using the structured interventional medical reporting data
g) outputting the clinical report.

Accordingly, a medical report may be quickly assembled from the data gathered during a medical intervention in the structured interventional medical reporting data.

According to an embodiment of the second aspect, a method is provided as described previously, further comprising after step c) the step c1) of:
c1) displaying, on a graphical user interface, a graphical input element linked to the template class element, wherein the graphical input element is selected from a plurality of graphical input elements, and

wherein in step e), the data is received into the template class element via the graphical input element.

Accordingly, it is possible to select images easily during a medical intervention procedure, and to include them in a template class element of structured interventional medical reporting data, which may be used to compose a medical report.

According to an embodiment of the second aspect, a method is provided as described above, further comprising the steps of:
f3) receiving a digital signature from an input device;
g) outputting the interventional clinical report incorporating the digital signature.

A system configured for medical reporting of an interventional medical procedure, comprising

an interventional medical equipment arrangement;

an apparatus according to the first aspect or its described embodiments, and

a storage device;

wherein the interventional medical equipment arrangement comprises an interventional procedure status monitoring device;

wherein the processing unit of the apparatus is communicatively coupled to the interventional procedure status monitoring device; and

wherein the processing unit is configured to store structured interventional medical reporting data from the apparatus onto the storage device.

According to a fourth aspect of the invention, a computer program element for controlling an apparatus an apparatus according to the previous first aspect, and its embodiments, is provided which, when the computer program element is executed by a processing unit, is adapted to perform the method steps according to the previous second aspect, and its embodiments.

According to a fifth aspect of the invention, a computer-readable medium having stored the program element of the fourth aspect is provided.

In this description, the term “interventional medical procedure” refers, for example, to an intravascular process undertaken on a patient such as a catheterization, a stent deployment, or a laparoscopy. Alternatively, the term refers to a procedure such as an endoscopy or colonoscopy. Optionally, the term involves a procedure in which imaging equipment is used to observe a patient's condition, such as a fluoroscope, or an item of ultrasound equipment.

In this description, the term “structured interventional medical reporting data” refers to a data record having a standardized format for containing information which frequently results from a medical intervention procedure. It will be understood, that standardized data elements and structures can be defined by technical standards, such as the technical standard for medical procedure report data “HL7 CDA”, with vocabulary encoded under a standard such as “SMOMED/CT”, or the ICD-9 or ICD-10. It will be understood that there are many forms for providing structured interventional medical reporting data, and many potential data formats. The data is typically stored in digital format on a hard disk drive, tape drive, solid state drive, and the like, on a server system or a PACS system, or in a “cloud” storage system.

In this description, the term “report template class” refers to a generic report template which is appropriate for use with a certain interventional procedure. Therefore, there may be a report template class for diagnostic procedures such as a right or a left heart catheterization, congenital disease angiography, left ventriculography or aortography, a peripheral arteriography, or a vascular arteriography. In addition, there may be a report template class for interventional operations, such as those required in an intervention for peripheral artery disease, valvuloplasty, defect closure, or congenital stenosis. It will be appreciated that diagnostic and interventional report template classes may be modular, and merged to provide an overall template with cooperating diagnostic and interventional elements. The report template class provides template class elements and template class rules as discussed below. Optionally, the term “report template class” represents “report template class data”, in which the report template class is stored as a data element on a data carrier.

In this description, the term “template class elements” defines an atomic data value to be input during an interventional procedure. Therefore, in a medical diagnostic intervention, the template class element could contain details of oxygen saturation (in percent), pressures, pressure gradients, haemoglobin content, and the like. Data is input into template class elements either automatically (from data collected from a digital interface of various items of medical equipment), or by a user (for example, via GUI elements such as a keypad interface or a drop-down menu), during a medical intervention. Optionally, the term “template class elements” represents “template class element data”, in which the template class elements are stored as a data element on a data carrier.

In this description, the term “template class rules” defines a set of rules which state at what stage of a medical procedure certain template class elements are available for data input. Therefore, at a certain point of a catheterization, it may be required to provide information on the type of guide wire or balloon devices in use. In contrast, this information might not need to be entered in a diagnostic phase of an intervention, because diagnostic parameters such as a blood pressure would be more useful. In this example, a template class rule would be provided which defined that intervention-based template class elements are not available for input during a diagnostic step. Optionally, the term “template class rules” represents “template class rule data”, in which the template class rules are stored as a data element on a data carrier. In the following description, the term “interventional procedure status indication” defines feedback from medical equipment, or a medical professional such as a physician or ancillary staff, which can be used by a processing unit to determine that a sequence of diagnostic or interventional events in an interventional medical procedure has occurred, or is occurring, or that a data input is required.

A broad range of parameters are included in the definition of the term interventional procedure status indication. The azimuth and elevation of a C-arm imaging device can be used to determine the plane of view of a heart during a cardiac intervention, for example. Therefore, a simple interventional procedure status indication could use the azimuth and elevation angle of a C-arm to define when a right heart ventricle or a left heart ventricle is being observed.

Alternatively, control signals from stent deployment devices could be used. A threshold monitoring of parameters, such as haemodynamic parameters or electrophysiological parameters could be used to provide the interventional procedure status indication. Image processing algorithms may be used to monitor the heart tissue to identify a contrast injection instant, or a balloon inflation instant.

The interventional procedure status indication could be as simple as a manual interventional procedure status input from a push-button signal present, from equipment being used during the intervention. It will also be appreciated that the interventional procedure status indication could be either a single signal, or a composite of many individual interventional procedure status indications, where the composite is defined by a set of interventional procedure status indication rules. For example, a left ventricular intervention procedure status indication rule may use a combination of the azimuth and elevation of the C-arm orientation, and a stent deployment control signal, to signify that a deployment of a stent during a left ventricular view has been completed.

Optionally, the term “interventional procedure status indication” represents “interventional procedure status indication data” which is received from the data signal output of an item of medical equipment used in an intervention.

In this description, it will be appreciated that the term “an interventional procedure status monitoring device” could be any device enabling the aforementioned interventional procedure status indication derivation. Thus, an interventional procedure status monitoring device could be a rotary encoder monitoring the C-arm elevation and azimuth quantities. Alternatively, the interventional procedure status monitoring device could be a stent deployment control signal monitoring unit. A wide variety of interventional procedure status monitoring devices will occur to the skilled person.

In an example, use may be made of generic data outputs of medical equipment providing information intended to be used for patient monitoring. The interventional procedure status monitoring device may be implemented on the same apparatus as used to implement the first aspect. In an example, the apparatus of the first aspect may be a general purpose computer, receiving the interventional procedure status indications as general purpose inputs. Assessment of the interventional procedure status would then be performed by the general purpose computer, based on the input signals.

In this description, the term “an availability condition” defines a quality of a template class element being made available for data input. In an example, if an availability condition of a template class element is satisfied, the template class element may be provided for display to a user, enabling the user to input information into the template class element at a certain procedural stage of a medical intervention procedure. Therefore, the availability condition may be derived by comparing the interventional procedure status indication to the template class rules.

Accordingly, the basic idea of the approach discussed herein, is to modulate the availability of a report template class as the need arises during phases of an interventional medical workflow, allowing the completion of only a relevant subset of the template class elements.

Therefore, the input of information into single or groups of template class elements at different times during the workflow is enabled. Only meaningful template class elements are selected for input at a certain instant of a medical procedure. There need not be a fixed order of display of the template class elements, and a selection of template class elements based on a current interventional step is possible.

These and other aspects of the present invention will become apparent from, and be elucidated with reference to, the embodiments described hereinafter.

### BRIEF DESCRIPTION OF THE DRAWINGS

Exemplary embodiments of the invention will be described with reference to the following drawings:

FIG. 1 shows a method according to the second aspect.

FIG. 2 shows an example of a data input process.

FIGS. 3a) and 3b) show example graphical input screens.

FIGS. 4a) and 4b) show variations of an interventional medical procedure.

FIG. 5 shows an apparatus according to the first aspect.

FIG. 6 shows a system configured for medical reporting according to the third aspect.

### DETAILED DESCRIPTION OF EMBODIMENTS

In the field of medical interventions, for example in a catheter lab, a physician has to fill out a patient report to document the findings and the outcome of the procedure. This is a time-consuming and error-prone task. The reporting is generally performed after the intervention. The physician therefore has to spend extra time on paperwork, or dictation. Information may be reported by logging equipment comprised in medical equipment such as ECG equipment, or from a PACS (Picture Archive and Communication System). To include such data in a post-procedure report implies that values of data recorded during an intervention would need to be transcribed from such equipment after the procedure, when less detail can be remembered. This increases the time required to write or dictate the report.

The required input for the data records of a template arises at different time instances of the workflow being followed, in an unpredictable order, in a typical intervention. However, current report templates require information to be filled in, in a linear format, in order of medical importance. Therefore, to fill in a conventional report template during a medical procedure would require a medical professional to jump back and forwards in the report, in order to fill in the various fields. This would not be user-friendly, and would disturb the physician during the intervention.

It is important to note also that a medical workflow is not necessarily the same each time, even for the same type of intervention. Because the workflow is not predictable in advance, and can take a different route, dependent events arising during an intervention, the selection of a different treatment option during an intervention, and even on the personal preferences of the physician, it is not predictable in advance when a certain type of data needs to be filled in to the template class.

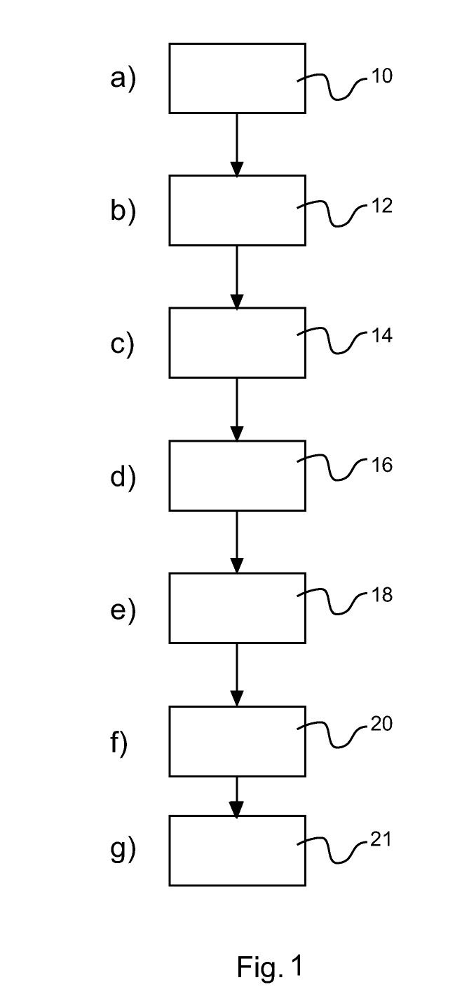

FIG. 1 shows a method for the formation of a clinical report of an interventional medical procedure using structured interventional medical reporting data, comprising the following steps:
a) receiving 10 a report template class of an interventional medical procedure,

wherein the report template class comprises a plurality of template class elements, and a plurality of template class rules,
b) receiving 12 an interventional procedure status indication from a procedure status monitoring device,

wherein the interventional procedure status indication provides information about a current stage of a plurality of stages of the interventional medical procedure;
c) determining 14 an availability condition of a template class element in the plurality of template class elements by comparing the interventional procedure status indication to at least one of the plurality of template class rules;
d) enabling 16 a data input into the template class element if the availability condition is satisfied;
e) receiving 18 data into the template class element for which data input has been enabled to form structured interventional medical reporting data; and
f) generating 20 a clinical report of the interventional medical procedure using the structured interventional medical reporting data
g) outputting 21 the clinical report.

Advantageously, the method according to this second aspect enables a clinical report to be generated using structured interventional medical reporting data assembled during an interventional medical procedure. An interventional procedure status indication is used to make a plurality of template class elements available (or unavailable) for data input. This means that the large and complex range of data input possibilities needed for a typical interventional medical report can remain hidden from a medical professional until a point at which they need to be provided in the interventional procedure.

In addition, the interventional procedure status indication may interact with the template class rules to enable a flexible display of the template class elements during a procedure, to enable data input to be customized to a flexible procedure order during an intervention. Thus, structured interventional medical reporting data may be input at meaningful time instances in an interventional medical workflow.

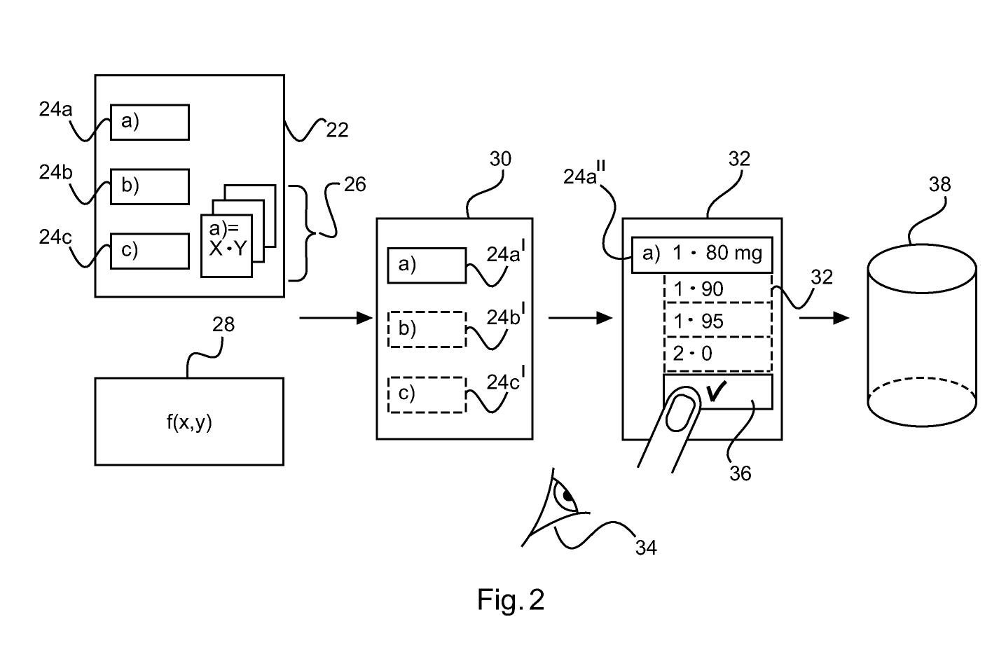

FIG. 2 shows a graphical example of this procedure. The report template class 22 is received, for example from a data carrier, such as a USB stick, a CD ROM, a DVD, or a computer network.

The report template class 22 may be viewed in an example as a blank, but structured data record enabling the input and storage of medical data. The report template class 22 comprises a plurality of template class elements 24a, 24b and 24c. The template class elements 24a, 24b and 24c are atomic data records, for storing parameters such as heart rate from an electrocardiogram, or an amount of anaesthetic delivered to a patient, or a blood flow parameter. “Atomic” in this sense means that the template class elements are for recording a unique observation at a certain time point, although an allowance can be made for later editing of the unique observation.

According to an example, records stored in the template class elements may be time-stamped when stored.

It will be appreciated that a wide range of parameters may be stored in the template class elements, for example sub-tables, or images. The report template class 22 also comprises a plurality of template class rules 26. In the example of FIG. 2, it is seen that the template class rule a) is in a “TRUE” condition when X AND Y are “TRUE”.

In other words, when the input parameters x and y are “TRUE”, the template class element 24a will be available for data input.

FIG. 2 also shows an interventional procedure status monitoring device 28 from which an interventional procedure status indication f(x,y) may be derived. It will be appreciated that a broad range of sources may be used to obtain the interventional procedure status indication. An interventional procedure status monitoring device can be considered as a simple piece of medical monitoring equipment, such as an electrocardiogram machine, or a balloon inflation monitor. The interventional procedure status monitoring device could be image-based, or even receive a separate input from another medical professional, such as a nurse involved in the interventional procedure.

FIG. 2 shows that the modified report class template 30 is modified when an availability condition of a template class element in the plurality of template class elements is assessed by comparing the interventional procedure status indication f(x,y) to one of the plurality of template class rules 26. In this case, f(x,y) satisfies rule A, and so the template class element a) 24a′ is displayed. However, the interventional procedure status indication does not enable the availability condition of template class elements b) or c) to be satisfied. Therefore, box 24b′ and box 24c′ are shown as dotted, which denotes that they are not available for user input. It will be appreciated that in an exemplary implementation with a GUI, box 24b′ and box 24c′ would either not be displayed to the user at all, or could be “greyed out”.

FIG. 2 shows the reception of data into template class elements of the report template class at 32. In this case, the template class element is in the form of a pre-populated drop down menu. An anaesthetic quantity value 1.80 mg is shown as having been pre-populated into the template class element 24a″. This information has been automatically pre-populated from an item of anaesthetic monitoring equipment monitoring anaesthetic dose.

Also shown in FIG. 2 is the ability to alter the value around the pre-populated value using a drop-down menu 32. A user 34 confirms the selection using confirmation box 36. At this point, the data received into the template class element as structured interventional medical reporting data may be stored in a storage device 38 for further use. In an example where the apparatus is implemented on a personal computer, the storage device may be the hard drive, or a server or cloud storage arrangement to which the personal computer is communicatively coupled.

Finally, a clinical report (not shown in FIG. 2) may be generated and output, based on the stored structured interventional medical reporting data. Reports of differing formats may be prepared for different people (reports for nurses, consultants, etc). According to an example, the report is provided as a .pdf document. According to an example, the report is printed using a printer. According to an example, the report is made available on a server for download, or published to an intranet page for future analysis.

Although FIG. 2 has been explained in terms of a graphical user interfaces and their interaction with the user, it will be appreciated that it is possible to fill data into a template class element according to template class rules also based on, for example, voice recognition software, and so the explanation of FIG. 2 is not limited to graphical user interfaces. In addition, it will be appreciated that FIG. 2 has been discussed in terms of one simple reporting operation, although more complex reporting operations are possible.

A specific discussion of an embodiment of the proposed method is now provided.

Firstly, an automatic intra-procedural analysis of a medical workflow, and the phases of the workflow, may provide a number of different cardiac intervention phases, such as a stenting phase or a valve placement phase. Rules defining the stages of possible diagnosis or intervention phases can be defined by a medical professional, or a software designer. When it is detected that an intervention phase which has to be documented is finished, template class elements of the reporting class to be filled in can be displayed, for example on a GUI. The medical workflow phases, can, for example, be identified based on existing data streams such as those from an electrocardiogram, C-arm angulation parameters, or imaging content.

A stenting procedure of a coronary tree would involve the acquisition of a number of two-dimensional images of the left and the right sides of the heart. These are acquired in order to identify coronary stenoses, and variants in the coronary tree.

When the end of the left heart side imaging is detected, the findings for the LAD, LCA, and Cₓ can be reported at that time point.

Thus, an interventional procedure status indication that the medical procedure is at this stage is reported, enabling an availability condition of a template class element for LAD (Left Anterior Descending artery), LCA (Left Coronary Artery), and Cₓ (Circumflex artery) to be made available for data input. Then, a similar procedure occurs for the measurement of the right heart side, and the RCA. Other important diagnostic steps, such as haemodynamics measurements, like pressure and waveform measurements, can be performed before and after the stenting. After these measurements are performed, the findings are held in structured interventional reporting data which may be used for a variety of purposes.

Thus, the imaging of a left heart side may enable, for example, a user interface to enable the input of a left coronary tree description.

Such a coronary tree description may be based on a simple “drop-down” menu selection of a medical indication for each relevant branch of an artery. Alternatively, the coronary tree description may be a generic 2D or 3D schematic of a coronary tree. Optionally, the coronary tree description can be generated from CT data of the patient under examination.

The C-arm may then be moved to allow imaging of the right heart side. The movement of a C-arm to a different azimuth and elevation provides an interventional procedure status indication enabling a right coronary tree description template class element to be enabled. Following this, pressure and waveform measurements may be input using a specific user interface for pre-interventional haemodynamics.

A stenting procedure is then carried out at stenoses adjudged to require treatment. A subsequent post-interventional haemodynamic input screen may be provided based on an interventional procedure status indication observing that a stent has been deployed. This observation may be from an image noticing a balloon expansion, or alternatively from a stent deployment signal.

At the end of an interventional step requiring documentation, a user interface may optionally be displayed, allowing the documentation of the findings of the current interventional step. Therefore, the information which is queried by the user interface should be limited to the current interventional step, and does not need to comprise the complete report template class.

Therefore, after imaging the left heart side, the user can document the left coronary tree. Information related only to the LAD, LCA, and Cₓ, and not the RCA is input.

For each of the coronary arteries, the type, and location, of the lesion and the stenosis degree have to be documented. In addition, variants that can occur in the left coronary tree, such as a third branch or a left dominant circulation can be documented, optionally using drop-down menus.

Apart from manual user input, information can also be obtained from other sources such as the imaging system, the electrocardiogram, and the pressure wire, and in an embodiment, can already be pre-filled in the user interface. For example, the stenosis degree, or location of the stenosis can be automatically extracted from fluoroscopy, and displayed in the user interface. Thereby, the user still has the change to manually correct these values by themselves.

The user interface may be filled in by a physician using a touchpad, or a nurse, who would be logging the procedure in a control room. Drop down lists in the user interface may enable the input to be limited to a standardized vocabulary, or lexicon, of concepts.

Information may be transferred into the report template, because the link between the information from different user interfaces shown in the intervention, and the report template is known. Therefore, after filling in information at different user interfaces (report template classes), information can be directly transferred into the report template automatically.

After an intervention, a physician can examine the filled in report template, and optionally complement it with additional values, or free-text, if necessary. Optionally, a report can be automatically created from the report template, and can be signed by the physician before they leave the room.

According to an embodiment of the second aspect, a method is provided as discussed in connection with the second aspect, further comprising after step c), the step c1):
c1) displaying, on a graphical user interface, a graphical input element linked to the template class element, wherein the graphical input element is selected from a plurality of graphical input elements, and wherein in step e), the data is received into the template class element via the graphical input element.

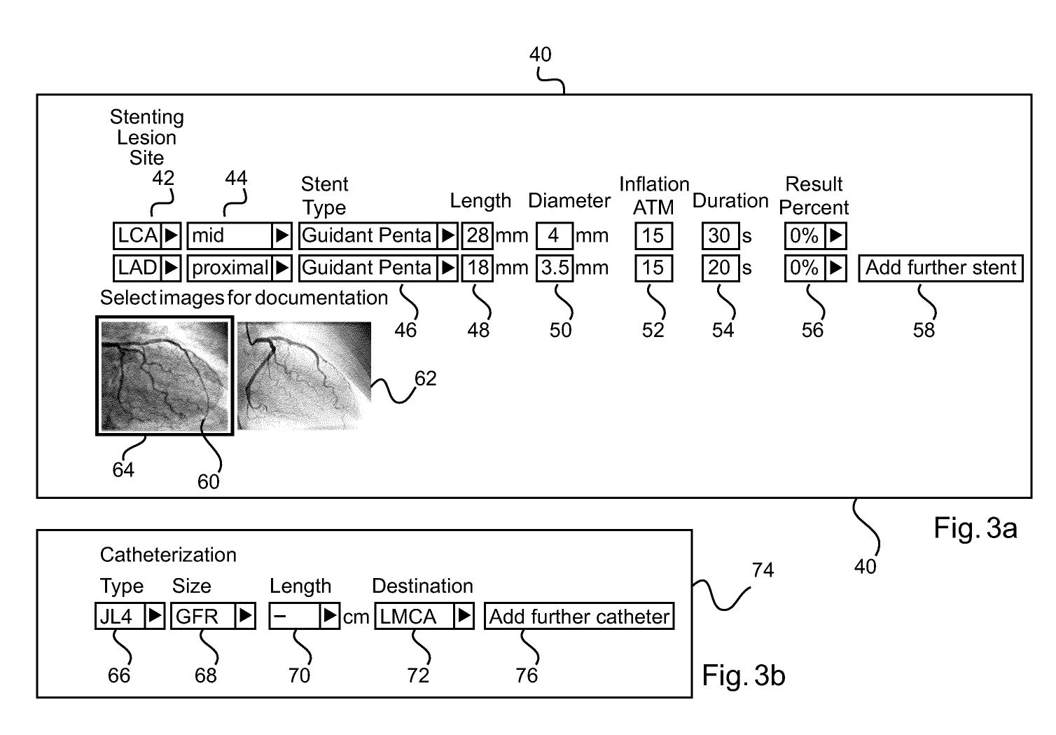

FIGS. 3a) and 3b) illustrate respectively a graphical user interface (GUI) for a stenting operation, and for a catheterization operation.

The stenting interface 40 comprises fields, comprising rows. Each row refers to an individual stent, as placed. For example, in FIG. 3a), two stents have already been placed, one at an LCA site, and one at a LAD site. Thus, there is a field enabling the input of information relating to the lesion site 42, the area on the coronary artery 44 where the lesion site may be viewed, fields defining the type of stent used 46, fields defining the length 48 and the diameter 50 of the stent, and parameters relating to the balloon inflation pressure 52 and duration 54. An assessment optimality result 56 may also be provided. There is an option 58 to provide a further stent if necessary. As can be seen, in an embodiment, it is possible to select an image 60 or 62 which has been obtained from an interventional medical imaging device, such as an angiogram. A selection boundary 64 is currently around the left-hand image 60, but by use of a cursor or a touch screen, a medical professional may select an alternative image.

FIG. 3b) shows an alternative graphical user interface for use in a catheterization operation. In this case, the type of catheter 66, the size of the catheter 68, the length of the catheter 70, and the destination of the catheter 72 may be input into the catheterization dialog 74. There is an option to add a further catheter at 76. In this example, the menus have been pre-populated from pre-interventional information.

Therefore, the use of graphical user interfaces enables template class information to be input to allow structured interventional medical reporting data to be composed using the same screens as used on medical intervention equipment.

According to an embodiment of the second aspect, a method is provided as described above, further comprising the steps of:
a1) receiving parameter information from a medical patient monitoring system; and
wherein step e) further comprises the steps of:
e1) pre-populating the graphical input element with an initial data value taken from the patient parameter information;
e2) previewing the initial data value of the template class element in the graphical input element; and
e3) receiving a user confirmation command confirming the initial data value as a final data value; and
f1) storing the final data value into the template class element as the structured interventional medical reporting data.

Therefore, as initially described in FIG. 3b), a pre-selected value for the template class element may be chosen. A pre-selected value could define stenosis location, or inflation pressure, for example. Optionally, input from a bar-code or QR code scan of a catheter in use could be used to select pre-selected values.

According to an embodiment, this value may be manually correctable in the GUI if necessary, using a keyboard, touchpad widget, or other data input means. The pre-selected value can be extracted or calculated from different sources such as the imaging of system parameters, electrocardiogram values, pressure measurements, medical images, and the like.

According to an embodiment of the invention, a method is provided according to the second aspect, further comprising the steps of:
a2) receiving standardized vocabulary data linked to the template class element, wherein the standardized vocabulary data comprises a plurality of vocabulary elements, and wherein each vocabulary element represents a standard medical term; and
c2) selecting a subset of vocabulary elements from the plurality of vocabulary elements based on the template class element;
c3) displaying a subset of vocabulary elements on the graphical user interface as the graphical input element; and
d1) enabling a selection of a selected vocabulary element, out of the subset of vocabulary elements;
e4) receiving the selected vocabulary element into the template class element.

Therefore, for the report template class, a standardized vocabulary of concepts may be used allowing an exchange of information without misinterpretations.

According to an embodiment of the second aspect, a method according to the second aspect is provided, further comprising the steps of
c4) storing a sequence of interventional procedure status indications;
c5) detecting the end of a current stage of the interventional medical procedure by comparing the sequence of interventional procedure status indications to an interventional procedure stage rule; and
c6) displaying the graphical input element on the graphical user interface if the end of the current stage of the interventional medical procedure has been detected.

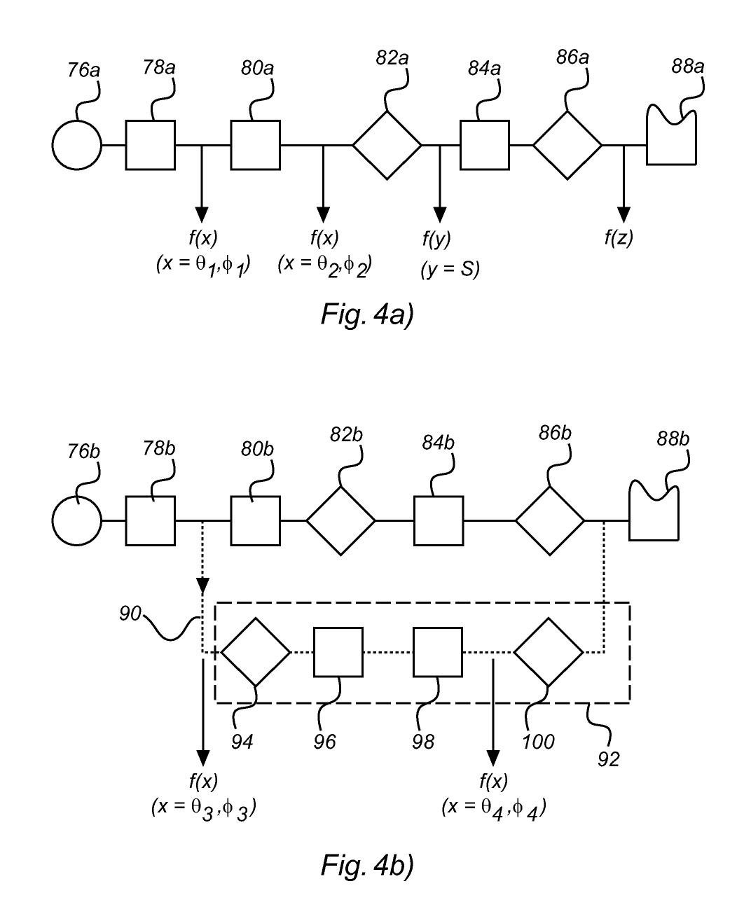

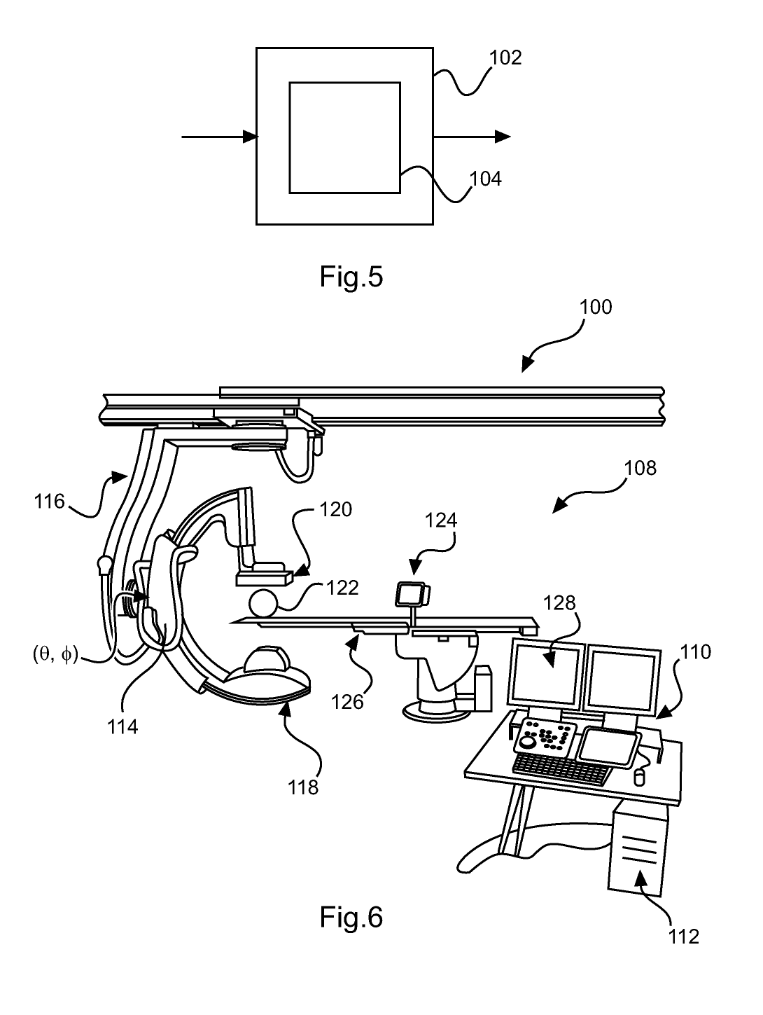

FIG. 4a) and FIG. 4b) show alternative interventional medical procedures in schematic format.

In these drawings, a circle represents an initializing data input stage, in which information relating to a patient preparation for a catheterization procedure is input. Such data could be the “time in”, “time out”, initial vital signs such as blood pressure or pulse, access site, anaesthesia support, surgical support, or haemodynamic support.

A square box represents an image processing or image capture operation performed, for example, using an angiography device.

Diamond-shaped boxes define a diagnostic procedure, such as a haemodynamic measurement, or the manual input of angiography findings.

Rhombus-shaped boxes define interventional procedures, such as equipment, baseline anatomy parameters, deployment of devices and their parameters, and intervention result assessments. The rectangular wave box represents the end of the procedure.

FIG. 4a), therefore, illustrates a simplified example of a heart stenting operation in which initial data is provided at 76a. A linear order for this operation may expect a left heart angiography 78a and a right heart angiography 80a to be performed using a C-arm.

It is noted, at this stage, that an interventional procedure status indication f(x) based on the azimuth and elevation of the C-arm changes from (θ₁; ϕ₁) to (θ₂; ϕ₂), implying that a different view of the heart is being taken. This may be used to change availability conditions of the images resulting from steps 78a and 80a.

At stage 82a, a diagnostic blood pressure measurement is made. Another logical interventional procedure status indication f(y) indicates that a stenting operation S is occurring. The variable y may be a binary balloon inflation signal, for example.

At step 84a, a stent has been deployed. At 86a, a post-interventional diagnostic blood pressure measurement f(z) is taken before an interventional procedure status indication indicates termination of the procedure, at 88a. The variable z may be an image detection of a catheter leaving the image frame, for example.

FIG. 4b) shows an alternative view of a measurement process which embodiments according to this aspect can address. At stage 76b, interventional initialization data is again input into the relevant report class templates. At 78b, 80b, 82b, 84b, 86b, and 88b, the same process as described in FIG. 4a) is provided. However, the entry of an alternative branch 90 may be detected if the interventional procedure status indication f(x), where x is the C-arm orientation, enters a condition from (θ₁; ϕ₁) to (θ₃; ϕ₃). This represents a clinician taking an alternative route through an intervention decision tree, perhaps owing to an interventional complication, or specific patient morphology issues.

Therefore, the dotted line box 92 and the procedures contained therein are representative of a sequence of interventional procedure status indications, which may be stored and detected to enable specific functionality of the template class elements as defined by the template class rules. Thus, in branch 90 inside box 92, a medical professional may opt to make a haemodynamic measurement 94 after the initial heart angiography 78b. A deployment may be made 96, detected as the logical interventional procedure status indication f(y), before the display of a right-hand side angiography 98 at a different C-arm position (θ₄; θ₄). Then, a second blood pressure measurement may be made after the right-hand side angiography at 100.

According to aspects discussed above, structural medical data may be composed using different clinical decision routes, as illustrated in FIGS. 4a) and 4b), by making the input of data into appropriate template class elements possible in a flexible order, as a different procedural route develops during an intervention.

According to an embodiment of the second aspect, a method is provided as described previously in relation to the second aspect, further comprising the steps of
a3) acquiring a first plurality of medical images of an interventional procedure from an interventional medical imaging device;
c7) displaying the plurality of medical images using the graphical user interface; and
d2) enabling a user selection of a medical image of the plurality of medical images; and
f2) storing user-selected selected medical images in the structured interventional medical reporting data.

Therefore, images will are already being used by the clinician to perform a medical intervention procedure may also be stored into the template report class appropriate as and when a useful image is seen. In this case, the graphical user interface could comprise a discreet sidebar option on the screen of the medical imaging equipment upon which the graphical user interface for data input is also displayed.

According to an embodiment of the second aspect, the method according to any preceding claim is provided further comprising after step c), the steps of
b1) receiving a second plurality of interventional medical images from an interventional medical imaging device;
d3) automatically selecting a representative image from the second plurality of interventional medical images based upon a medical image selection algorithm to provide a selected representative interventional image; and

wherein, in step e) enabling the data input into the template class element comprises enabling the input of the selected interventional representative image into the template class element.

According to this embodiment, an interventional image is automatically selected from a sequence of images recorded in an interventional medical imaging device. An image processing algorithm (as would be known to a person skilled in the art) allows the selection of a representative image from s second plurality of medical images providing, for example, an optimized aspect ratio, or an optimized view of a stent or a deployed balloon. This removes from a medical professional the burden of having to manually scroll through a sequence of images during or after the intervention.

According to an embodiment of the second aspect, a method is provided as described previously, wherein, in step b), the interventional procedure status indication is a signal resulting from the group of signals: (i) a stent deployment control signal; (ii) a stent deployment visual detection signal; (iii) a device positioning recognition signal; (iv) a haemodynamic measurement stage signal; (v) an electrophysio logical measurement stage signal; (vi) a pressure measurement signal; (vii) a contrast injection signal; (viii) a medical image state signal; and (ix) an electrocardiogram signal; (x) a balloon inflation signal; (xi) a C-arm orientation parameter; (xii) a manual interventional procedure status input; and (xiii) a catheter positioning recognition signal.

It will be appreciated that the interventional procedure status indication may be comprised of a single element (i) to (xii), of any combination of the elements (i) to (xiii).

Signals (ii), (iii), (vii), (viii), and (x), may, optionally, be detected using an image processing algorithm applied to the image sequence output from the C-arm imaging equipment.

Signals (i), (vii), (x), and (xii) may, optionally, be taken from logical commands input to medical equipment to provide for the respective stent deployment, contrast injection, balloon inflation, etc.

Signals (iv), (v), (vi), and (ix) may, optionally, be obtained from information communication interfaces of medical equipment measuring the relevant quantities.

Signal (xi) may, optionally, be obtained from rotary encoders present in the mechanical system of a C-arm arrangement.

The medical image state signal may, optionally, be provided by an image processing algorithm detecting the presence of an imaging condition. For example, a ridge filter could be used to determine the presence of a catheter in the field of view, or not.

The medical image state signal may, optionally, be provided by a signal indicating that a certain image “snapshot” is being taken.

Therefore, the interventional procedure status indication can be derived from one of the proceeding signals, or a combination of them, according to a rule defined in the template class rule. Thus, an example interventional procedure status indication would combine prior diagnostic knowledge that stenoses were present in the LCA, LAD, and RCA, and an interventional procedure status indication that left heart imaging was being performed. Thus, a set of template classes enabling the input of information about balloon inflation operation could be unlocked for the input of information into a report class template, but only in relation to the LCA and the LAD. Listing of input options for the RCA would be superfluous at this stage.

Of course, the skilled person could provide other interventional procedure status indications, and compose template class rules from them.

According to an embodiment of the second aspect, there are provided the further steps of:
f3) receiving a digital signature from an input device;
g) outputting the clinical report incorporating the digital signature.

Accordingly, an interventional medical procedure report may be generated substantially instantaneously following the end of an interventional medical procedure, containing verifiably accurate medical information.

Optionally, the medical procedure report may be output as an encrypted .pdf (TM) document, a signed video, or an encrypted web form, The medical procedure report may be displayed to the medical professional before generation, enabling alteration of values before final signing of the report.

According to a first aspect, there is provided an apparatus 102 for the formation of a clinical report during an interventional medical procedure using structured interventional medical reporting data. The apparatus comprises:

a processing unit 104;

wherein the processing unit 104 is configured to receive a report template class of an interventional medical procedure,

wherein the report template class comprises a plurality of template class elements, and a plurality of template class rules,

to receive an interventional procedure status indication from an interventional procedure status monitoring device;

wherein the interventional procedure status indication provides information about a current stage of a plurality of stages of the interventional medical procedure,

to determine an availability condition of a template class element in the plurality of template class elements by comparing the interventional procedure status indication to one of the plurality of template class rules, to enable a data input into the template class element if the availability condition is satisfied, to receive data into the template class element for which data input has been enabled to form structured interventional medical reporting data, to generate a clinical report using structured interventional medical reporting data, and to output the clinical report.

According to an embodiment of the first aspect, an apparatus 102 is provided as described in accordance with the first aspect, wherein the processing unit 104 is further configured to display, on a graphical user interface, a graphical input element linked to the template class element, wherein the graphical input element is selected from a plurality of graphical input elements, and wherein the data is received into the template class element via the graphical input element.

According to an embodiment of the first aspect, an apparatus 102 is provided, wherein the processing unit 104 is further configured to receive patient parameter information from a patient monitoring device, to pre-populate the graphical input element with an initial data value taken from the patient parameter information, and to preview the initial data value of the template class element in the graphical input element, to receive a user confirmation command confirming the initial data value as a final data value; and to store the final data value into the template class element as structured interventional medical reporting data.

According to an embodiment of the first aspect, the apparatus 102 is provided, wherein the processing unit 104 is further configured to receive standardized vocabulary data linked to the template class element; wherein the standardized vocabulary data comprises a plurality of vocabulary elements, and wherein each vocabulary element represents a standard medical term; and to select a subset of vocabulary elements from the plurality of vocabulary elements based on the template class element, to display a subset of vocabulary elements on the graphical user interface as the graphical input element, and to enable a selection of a selected vocabulary element, out of the subset of vocabulary elements, and to receive the selected vocabulary element into the template class element.

According to an embodiment of the first aspect, the apparatus 102 is provided, wherein the processing unit 104 is further configured to store a sequence of interventional procedure status indications, to detect the end of a current stage of the interventional medical procedure by comparing the sequence of interventional procedure status indications to an interventional procedure stage rule, and to display the graphical input element on the graphical user interface if the end of the current stage of the interventional medical procedure has been detected.

According to an embodiment of the first aspect, there is provided an apparatus 102, wherein the processing unit 104 is further configured to acquire a first plurality of medical images of an interventional procedure from an interventional medical imaging device, to display the plurality of medical images using the graphical user interface; to enable a user selection of an interventional medical image of the plurality of medical images, and to store the user-selected medical images in the structured interventional medical reporting data.

According to an embodiment of the first aspect, an apparatus 102 is provided as described previously, wherein the processing unit 104 is further configured to receive second plurality of interventional medical images from an interventional medical imaging device, and to automatically select a representative image from the second plurality of interventional medical images based upon an interventional medical image selection algorithm to provide a selected representative interventional image, wherein a data input into the template class element comprises enabling the input of the selected representative image into the template class element.

According to an embodiment of the first aspect, an apparatus 102 is provided according to the previous description, wherein the interventional procedure status indication is a signal received by the processing unit 104 resulting from the group of signals: (i) a stent deployment control signal, (ii) a stent deployment visual detection signal, (iii) a device positioning recognition signal, (iv) a haemodynamic measurement stage signal, (v) an electrophysio logical measurement stage signal, (vi) a pressure measurement signal, (vii) a contrast injection signal, (viii) a medical image state signal, (ix) an electrocardiogram signal, (x) a balloon inflation signal, (xi) a C-arm orientation parameter, (xii) a manual procedure status input.

According to an embodiment of the first aspect, an apparatus is provided as previously described, further configured to receive a digital signature from an input device and to output the clinical report incorporating the digital signature.

According a third aspect of the invention, there is provided a system 106 configured for medical reporting of an interventional medical procedure. The system comprises:

An interventional medical equipment arrangement 108,

an apparatus 110 according to the previous description of the first aspect or any of its embodiments, and a storage device 112.

The medical intervention arrangement comprises an interventional procedure status monitoring device 114. The apparatus 110 is communicatively coupled to the interventional procedure status monitoring device 114. The apparatus 110 is configured to store structured interventional medical reporting data in the storage device 112.

FIG. 6 shows a system configured for interventional medical reporting. In FIG. 6, the system 106 comprises a interventional medical equipment arrangement 108 having a C-arm 116 comprising an X-ray source 118 and an X-ray detector 120. Such a system may operate as an angiography device. An object of interest 122, such as a patient, is positioned in between the path of radiation between the X-ray source 118 and the X-ray detector 120.

According to an example, the C-arm 116 may be used to derive an interventional procedure status indication 114, in this case the azimuth and elevation (θ,ϕ) of the C-arm. In this example, as the C-arm is moved around the object of interest 122, a data input into different template class elements would be provided at different C-arm orientations.

According to an embodiment, a small user input touch screen 124 may enable a medical professional to input information into the template report class via a graphical user interface without leaving the table 126. Alternatively, another medical professional, such as a nurse, may input information into the report class using the remote computer 128.

According to a fourth aspect, a computer program element for controlling an apparatus according to one of the previous embodiments is provided which, when the computer program element is executed by a processing unit, is adapted to perform the method steps according to the previously described method steps.

According to a fifth aspect, a computer-readable medium having stored the program element of the fourth aspect is provided.

A computer program element might therefore be stored on a computer unit, which might also be part of an embodiment of the present invention. This computing unit may be adapted to perform or induce performance of the steps of the method described above.

Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both the computer program that has the invention installed from the beginning, and a computer program that by means of an update turns an existing program into a program that uses the invention. A computer program may be stored and/or distributed on a suitable medium, such as an optical storage media or a solid state medium supplied together with, or as a part of other hardware, but may also be distributed in other forms, such as via the Internet or other wired or wireless telecommunication systems.

However, the program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It should to be noted that embodiments of the invention are described with reference to different subject-matters. In particular, some embodiments are described with reference to method-type claims, whereas other embodiments are described with reference to the device-type claims. However, a person skilled in the art will gather from the above, and the following description, that unless otherwise notified, in addition to any combination of features belonging to one type of subject-matter, also any other combination between features relating to different subject-matters is considered to be disclosed with this application.

All features can be combined to provide a synergetic effect that is more than the simple summation of the features.

While the invention has been illustrated and described in detail in the drawings and foregoing description, such illustration and description are to be considered illustrative or exemplary, and not restrictive. The invention is not limited to the disclosed embodiments.

Other variations to the disclosed embodiments can be understood, and effected by those skilled in the art in practicing the claimed invention, from a study of the drawings, the disclosure, and the dependent claims.

In the claims, the word “comprising” does not exclude other elements or steps, and the indefinite article “a” or “an” does not exclude a plurality. A single processor, or other unit, may fulfil the functions of several items recited in the claims. The mere fact that certain measures are recited in mutually different dependent claims does not indicate that a combination of these measures cannot be used to advantage. Any reference signs in the claims should not be construed as limiting the scope.

1. An apparatus for the formation of a clinical report during an interventional medical procedure using structured interventional medical reporting data, the apparatus comprising:
a processing unit;
wherein the processing unit is configured to receive a report template class of an interventional medical procedure,
wherein the report template class comprises a plurality of template class elements, and a plurality of template class rules,
to receive an interventional procedure status indication from an interventional procedure status monitoring device;
wherein the interventional procedure status indication provides information about a current stage of a plurality of stages of the interventional medical procedure,
to determine an availability condition of a template class element in the plurality of template class elements by comparing the interventional procedure status indication to one of the plurality of template class rules, to enable a data input into the template class element if the availability condition is satisfied, to receive data into the template class element for which data input has been enabled to form structured interventional medical reporting data, to generate a clinical report of the interventional medical procedure using structured interventional medical reporting data, and to output the clinical report.

2. The apparatus according to claim 1,
wherein the processing unit is further configured to display, on a graphical user interface, a graphical input element linked to the template class element, wherein the graphical input element is selected from a plurality of graphical input elements, and
wherein the data is received into the template class element via the graphical input element.

3. The apparatus according to claim 2,
wherein the processing unit is further configured to receive patient parameter information from a patient monitoring device, to pre-populate the graphical input element with an initial data value taken from the patient parameter information, and to preview the initial data value of the template class element in the graphical input element, to receive a user confirmation command confirming the initial data value as a final data value, and to store the final data value into the template class element as the structured medical reporting data.

4. The apparatus according to claim 2,
wherein the processing unit is further configured to receive standardized vocabulary data linked to the template class element,
wherein the standardized vocabulary data comprises a plurality of vocabulary elements, and wherein each vocabulary element represents a standard medical term, and
to select a subset of vocabulary elements from the plurality of vocabulary elements based on the template class element, to display a subset of vocabulary elements on the graphical user interface as the graphical input element, to enable a selection of a selected vocabulary element, out of the subset of vocabulary elements, and to receive the selected vocabulary element into the template class element.

5. The apparatus according to claim 2,
wherein the processing unit is further configured to store a sequence of interventional procedure status indications, to detect the end of a current stage of the interventional medical procedure by comparing the sequence of interventional procedure status indications to an interventional procedure stage rule, and to display the graphical input element on the graphical user interface if the end of the current stage of the interventional medical procedure has been detected.

6. The apparatus according to claim 2,
wherein the processing unit is further configured to acquire a first plurality of medical images of an interventional procedure from an interventional medical imaging device, to display the plurality of medical images using the graphical user interface, to enable a user selection of an interventional medical image of the plurality of medical images, and to store the user-selected selected interventional medical image in a template class element of the structured medical reporting data.

7. The apparatus according to claim 1,
wherein the processing unit is further configured to receive a second plurality of interventional medical images from an interventional medical imaging device, and to automatically select a representative image from the second plurality of medical images based upon an interventional medical image selection algorithm to provide a selected representative interventional image,
wherein a data input into the template class element comprises enabling the input of the selected interventional representative image into the template class element.

8. The apparatus according to claim 1,
wherein the interventional procedure status indication is a signal received by the processing unit resulting from the group of signals:
(i) a stent deployment control signal;
(ii) a stent deployment visual detection signal;
(iii) a device positioning recognition signal;
(iv) a haemodynamic measurement stage signal;
(v) an electrophysiological measurement stage signal;
(vi) a pressure measurement signal;
(vii) a contrast injection signal;
(viii) a medical image state signal;
(ix) an electrocardiogram signal;
(x) a balloon inflation signal;
(xi) a C-arm orientation parameter;
(xii) a manual procedure status input; and
(xiii) a catheter positioning recognition signal.

9. The apparatus according to claim 1,
wherein the processing unit is further configured to generate an interventional medical procedure report using the structured medical reporting data by transferring the data stored in the plurality of template class elements into the report template class; and to output the interventional medical procedure report.

10. A method for the formation of a clinical report of an interventional medical procedure using structured interventional medical reporting data, comprising the following steps:
a) receiving a report template class of an interventional medical procedure,
wherein the report template class comprises a plurality of template class elements, and a plurality of template class rules,
b) receiving an interventional procedure status indication from a procedure status monitoring device,
wherein the interventional procedure status indication provides information about a current stage of a plurality of stages of the interventional medical procedure;
c) determining an availability condition of a template class element in the plurality of template class elements by comparing the interventional procedure status indication to at least one of the plurality of template class rules;
d) enabling a data input into the template class element if the availability condition is satisfied;
e) receiving data into the template class element for which data input has been enabled to form structured interventional medical reporting data; and
f) generating a clinical report of the interventional medical procedure using the structured interventional medical reporting data;
g) outputting the clinical report.

11. The method according to claim 10, further comprising after step c) the step c1) of:
c1) displaying, on a graphical user interface, a graphical input element linked to the template class element, wherein the graphical input element is selected from a plurality of graphical input elements, and
wherein in step e), the data is received into the template class element via the graphical input element.

12. The method according to claim 10, further comprising the steps of:
f3) receiving a digital signature from an input device;
g) outputting the clinical report incorporating the digital signature.

13. A system configured for medical reporting of an interventional medical procedure, comprising:
an interventional medical equipment arrangement;
an apparatus according to claim 1, and
a storage device;
wherein the interventional medical equipment arrangement comprises an interventional procedure status monitoring device;
wherein the processing unit of the apparatus is communicatively coupled to the interventional procedure status monitoring device; and
wherein the processing unit is configured to store structured interventional medical reporting data from the apparatus onto the storage device.

14. A computer program element for controlling an apparatus according to claim 1 which, when the computer program element is executed by a processing unit, is adapted to perform method steps.

15. A computer readable medium having stored the program element of claim 14.
